(19) United States
(12) Patent Application Publication
(10) Pub. No.: US 2006/0057062 A1
(43) Pub. Date: Mar. 16, 2006

(54) Compositions and methods useful in pretargeted imaging

(76) Inventor: Dinko Gonzalez Trotter, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 10/937,433

(22) Filed: Sep. 10, 2004

(51) Int. Cl. A61K 51/00; C07K 16/46

(52) U.S. Cl. 424/1.49; 530/391.1

(57) ABSTRACT

Disclosed are multispecific macromolecular constructs, blocking agents and radiolabeled effector molecules, as well as kits and methods for imaging tissue of interest in a mammalian subject. The multispecific macromolecular construct is capable of binding a radiolabeled effector molecule that can be imaged, as well as a disease marker such as for example a tumor specific antigen expressed on the surface of tumor tissue. The blocking agent comprises, or alternatively consists of, an unlabeled form of the radiolabeled effector conjugated to a carrier protein or polypeptide, said carrier protein or polypeptide preferably being non-immunogenic or having low immunogenicity. The invention further contemplates methods of imaging diseases or disorders in a mammalian subject using said compositions

### FIELD OF THE INVENTION

The invention relates to labeled and unlabeled compositions and methods useful in the pretargeted imaging of diseases, disorders and conditions.

### BACKGROUND OF THE INVENTION

Imaging of diseases and other targeted tissue in animal models and the human body is an area of intense investigation. Numerous techniques exist for imaging various agents that have localized to targeted tissue, including, for example, single photon emission computed tomography (SPECT), magnetic resonance imaging (MRI), ultrasonic imaging, and the like. Another imaging technology, positron emission tomography (PET) is a high sensitivity, high resolution, non-invasive, imaging technique for the visualization of human disease. In PET, 511 keV photons produced during positron annihilation decay are detected. In the clinical setting, fluorine-18 (F-18) is one of the most widely used positron-emitting nuclides. F-18 has a half-life (t_{1/2}) of 110 minutes, and emits β+ particles at an energy of 635 keV, in 97% abundance.

The short half-life of some radionuclides such as F-18 has limited or precluded their use with high molecular weight probes including antibodies, antibody fragments, recombinant antibody constructs and high molecular weight receptor-targeted peptides. This is because these high molecular weight probes require many hours to days to equilibrate with their target and clear from background before a satisfactory image can be obtained. During this time, typical doses of F-18 would decay to levels that could not be imaged.

This problem can be addressed by using other positron-emitting radionuclides, such as Cu-64, Br-76 and I-124, with much longer half-lives instead of the shorter lived radionuclides, like F-18. However, these longer-lived radionuclides are not as advantageous as F-18 for several reasons. The positron energy of F-18 is lower than other longer-lived radionuclides and thus F-18 can provide images with superior resolution. F-18 decays primarily via positron emission while the longer-lived radionuclides decay via multiple pathways; only positron emission is useful for imaging. Thus subjects must receive a large dose of radiopharmaceutical and endure more substantial radiation exposure to obtain an acceptable image. For some longer-lived radioisotopes, alternative decay pathways can produce emissions that interfere with collection of photons from positron annihilation; this complicates the process for obtaining a suitable image. F-18 is commercially more readily available than the longer-lived radionuclides. The longer half-life invariably means that the subject will receive a larger radioactive dose. Lastly, F-18 can be easily inserted into biologically active molecules (ligands) that target biomarkers that are associated with disease. Sterically, F-18 resembles a proton and insertion of a F-18 for a proton often does not affect biological activity. Longer-lived radionuclides are sterically more demanding (such as Br-76 or I-124) or require chelates (Cu-64) to remain affixed to the ligand; often incorporation of these longer-lived radionuclides impairs the targeting capability of these ligands.

Diseases and disorders, such as cancers for example, can be treated and diagnosed by directing to the diseased tissue antibodies or antibody fragments that are capable of targeting a diagnostic agent or therapeutic agent to the disease site. One approach to this methodology utilizes bi-specific monoclonal antibodies (bsAbs) having at least one binding site directed against a targeted diseased tissue and an additional binding site directed against a low molecular weight effector. This method includes administering a bsAb, allowing it to localize to the target and to clear normal tissue, and then administering a radiolabeled low molecular weight effector that is recognized by the second binding site of the bsAb. The radiolabeled low molecular weight effector also localizes to the original target.

The bsAb/low MW effector system has other considerations. First, the binding site of the bsAb having specificity for the low MW effector must bind with high affinity, since a low MW effector is designed to clear the living system rapidly if not bound by the bsAb. Second, it is desirable that the non-bsAb-bound low MW effector clear the living system rapidly to avoid non-target tissue uptake and retention. Third, the detection and/or therapy agent must remain associated with the low MW effector throughout its application within the bsAb protocol employed.

Thus, bispecific antibodies have been proposed that direct molecular complexes to cancers and other diseased tissue using antibodies of appropriate dual specificity. The molecular complexes used are often radioactive, using radionuclides such as cobalt-57 (Goodwin et al., U.S. Pat. No. 4,863,713), indium-111 (Barbet et al., U.S. Pat. Nos. 5,256,395 and 5,274,076, Goodwin et al., J. Nucl. Med. 33:1366-1372 (1992), and Kranenborg et al. Cancer Res (suppl.) 55:5864s-5867s (1995) and Cancer (suppl.) 80:2390-2397 (1997)) and gallium-68 (Boden et al., Bioconjugate Chem. 6:373-379, (1995) and Schuhmacher et al. Cancer Res. 55:115-123 (1995)) for radioimmunoimaging. Most of these bispecific antibodies and molecular complexes are described as useful in therapeutic applications. However, these compositions are not known to be particularly useful in conjunction with short-lived radionuclides in imaging applications.

The description herein of problems and disadvantages associated with known products, methods, and apparatus is not intended to limit the invention to the exclusion of these known entities. Indeed, embodiments of the invention may include some or all of the known products, methods, and apparatus without suffering from some of the problems and disadvantages described herein.

### BRIEF SUMMARY OF THE INVENTION

There remains a need for compositions and methods useful in generating imaging complexes with short-lived radionuclides in imaging applications. Embodiments of the invention address the need for compositions and methods useful in generating imaging complexes with short-lived radionuclides in imaging applications, by providing compositions and methods that help to improve the target-to-background ratio of pretargeted imaging techniques.

Features of embodiments of the invention are directed to compositions and methods that are useful in the diagnosis, imaging and detection of diseases, disorders and conditions of the human body, particularly diseases such as cancer. More generally, diseases, disorders and conditions may be diagnosed, imaged or detected in which one or more distinct disease markers representing the presence of the disease state are capable of being bound by multispecific macromolecular constructs. Such markers may, for example, be expressed on the cell surface of a target tissue.

The compositions and methods of the invention are further useful in the development and use of preclinical animal models in which one or more distinct markers representing a disease, disorder or condition are capable of being bound by multispecific macromolecular constructs and thereby diagnosed, imaged or detected. In a preferred embodiment of the invention, the animal models are based on murine or rat models of a disease or disorder.

In certain embodiments of the invention, the composition useful for this imaging, detection and diagnosis of diseases, disorders or conditions comprises, or alternatively consists of, one or more of the following components:

1. a multispecific macromolecular construct having specificity for an antigenic determinant of a disease that is preferably expressed on the surface of, for example, a diseased cell, the multispecific macromolecular construct also having specificity for an effector molecule;

2. a blocking agent that comprises, or alternatively consists of, an unlabeled effector molecule identical in structure to the radiolabeled effector molecule, conjugated to a carrier such as a polypeptide or protein, modified or unmodified DNA/RNA strands, or single strands thereof; and

3. an injectable, radiolabeled effector molecule that is useful as an imaging agent.

In accordance with other features of embodiments of the invention, there is provided a method of diagnosing, detecting or imaging a disease or disorder comprising, or alternatively consisting of, administering to a subject a multispecific macromolecular construct capable of binding to an antigenic determinant expressed by the diseased tissue, as well as capable of binding to an effector molecule. A sufficient period of time is allowed to pass in which the multispecific macromolecular construct localizes on the diseased tissue. Following the period of time provided to allow for the localization of the multispecific macromolecular construct on the diseased tissue, a blocking agent may be administered to the subject and provided a sufficient period of time to allow the blocking agent to bind to circulating multispecific macromolecular construct. Following this period of time, the radiolabeled effector is administered to the subject, and the targeted tissue is imaged with techniques known in the art. One such imaging technique known in the art is positron emission tomography.

In another feature of an embodiment of the invention, a kit is provided that includes a plurality of containers having the multispecific macromolecular construct, the blocking agent, and the radiolabeled effector separately placed within the plurality of containers, and instructions for administration thereof to a subject.

In accordance with these and other features of various embodiments of the invention, there is provided a radiolabeled effector having the formula:
La-l-R
where La is a short-lived labeling agent, l is a linking ligand capable of linking La to R, and R is an effector molecule capable of being bound by a multispecific macromolecular construct, said multispecific macromolecular construct also being capable of binding a disease marker associated with a tissue of interest.

In accordance with another feature of an embodiment of the invention, there is provided a blocking agent having the formula:
C-l-R
where C is a carrier such as a protein, polypeptide, DNA/RNA, DNA/RNA analog or chimeric construct, l is a linking ligand capable of linking C to R, and R is an unlabeled effector molecule that is recognized or capable of being bound by a multispecific macromolecular construct. Preferably the carrier C is a non-immunogenic or low immunogenicity carrier.

### BRIEF DESCRIPTION OF THE FIGURES

FIG. 1 shows the macromolecular construct (MMC) in circulation binding to the blocking agent, thus preventing binding of the labeled effector molecule to the MMC.

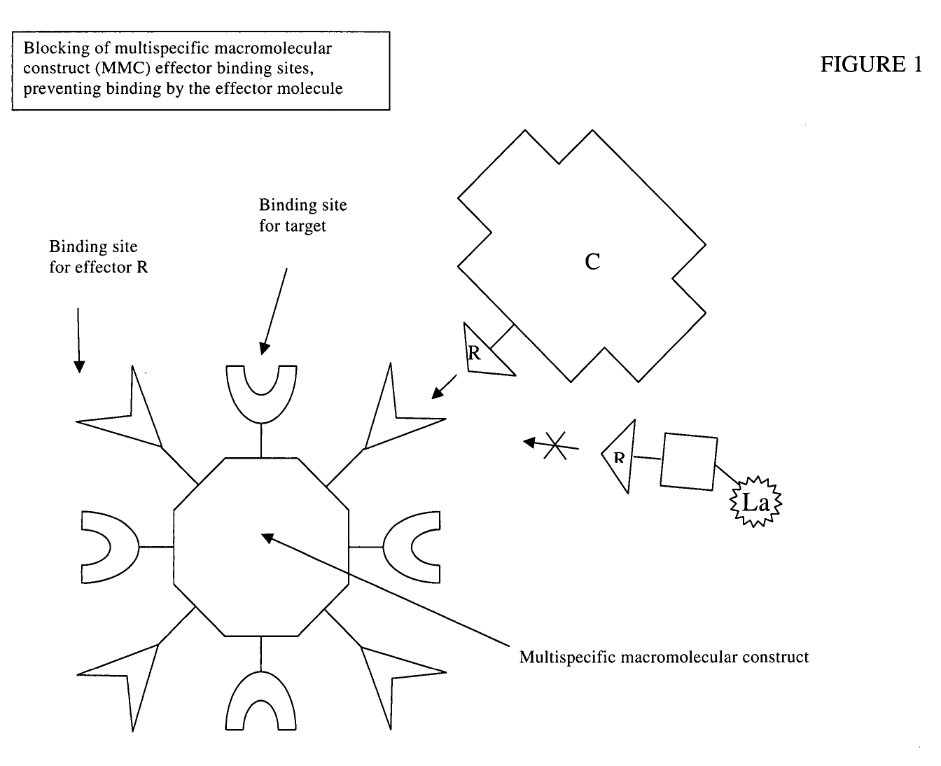

FIG. 2 shows the macromolecular (MMC) construct binding to the target and the labeled effector molecule binding to the MMC.

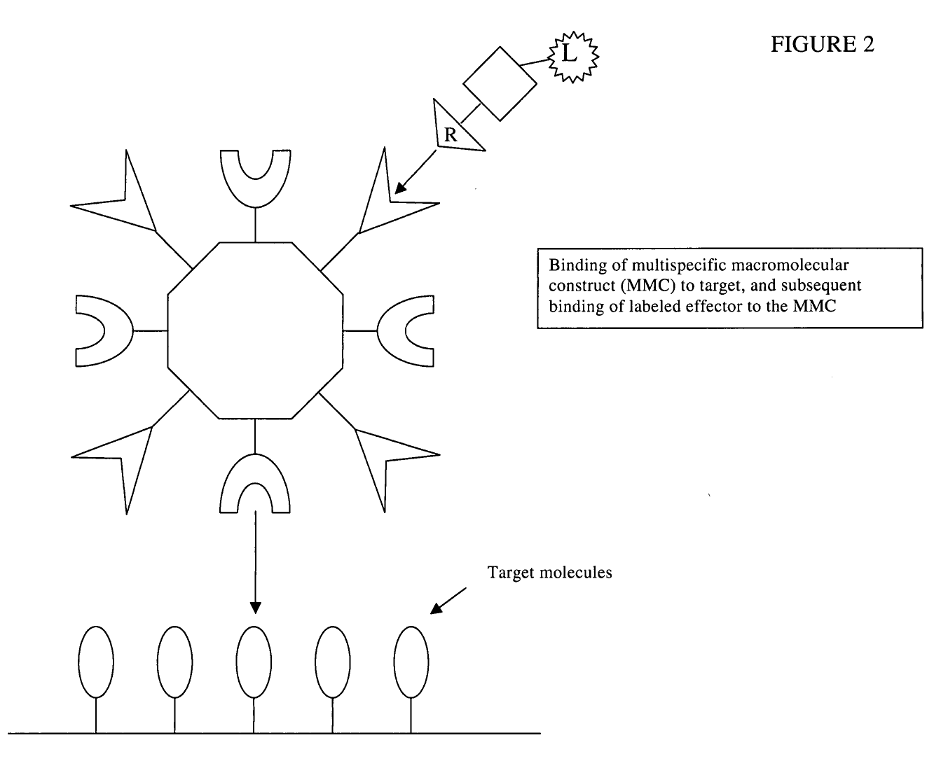

### DETAILED DESCRIPTION OF PREFERRED EMBODIMENTS

Before embodiments of the present compositions and methods are described, it is understood that this invention is not limited to the particular methodology, protocols, cell lines, vectors, and reagents described, as these may vary. In addition, the terminology used herein is for the purpose of describing particular embodiments only, and is not intended to limit the scope of the present invention which will be limited only by the appended claims.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the compounds, molecules, cell lines, vectors, and methodologies that are reported in the publications and that might be used in connection with the invention. Nothing herein is to be construed as an admission that the invention is not entitled to antedate such disclosure by virtue of prior invention.

As used herein, the term “multispecific macromolecular construct” refers to high molecular weight molecules capable of binding to both an antigenic determinant that functions as a disease marker as well as to an effector, either a radiolabeled effector or an unlabeled effector conjugated to a carrier, the multispecific macromolecular construct optionally capable of binding to one or more additional molecules including but not limited to antigenic determinants. In a preferred embodiment of the invention, multispecific macromolecular constructs include, but are not limited to, bispecific antibodies.

As used herein, the term “antibody” refers to intact molecules as well as to fragments thereof, such as Fab, F(ab′)2, and Fv fragments, which are capable of binding the antigenic determinant. “Antibody” also denotes variants, derivatives, and peptide mimetics of the disclosed antibody. Antibodies that bind the tissue of interest can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide then is used to immunize the animal.

As used herein, the term “effector” refers to a molecule that binds to the multispecific macromolecular complex with specificity. As used herein, “specificity” refers to an affinity of at least about K_{D}=100 nM. Furthermore, a carrier molecule or a signal-producing moiety (such as a PET isotope) may be bound to the effector molecule.

The term “antigenic determinant,” as used herein, refers to that fragment of a molecule (i.e., an epitope) that makes contact with a particular antibody. When a protein or a fragment of a protein is used to immunize a host animal, numerous regions of the protein may induce the production of antibodies which bind specifically to antigenic determinants (given regions or three-dimensional structures on the protein). An antigenic determinant may compete with the intact antigen (i.e., the immunogen used to elicit the immune response) for binding to an antibody.

A “composition comprising a given molecule” (e.g., multispecific macromolecular construct, antibody, bispecific antibody, or diabody) or a “composition comprising a given amino acid sequence,” as these terms are used herein, refer broadly to any composition containing the given molecule, amino acid sequence or polynucleotide encoding the same. The composition may comprise a dry formulation, an aqueous solution, or a sterile composition. The compositions may be stored in freeze-dried form and may be associated with a stabilizing agent such as a carbohydrate. In hybridizations and other applications, the compositions may be deployed in an aqueous solution containing salts, e.g., NaCl, detergents, e.g., sodium dodecyl sulfate (SDS), and other components, e.g., Denhardt's solution, dry milk, salmon sperm DNA, etc.

Throughout this description, the expressions “specific binding” or “specifically binding,” “binding,” “binds,” and/or “recognizes” refer to the interaction between a molecule, protein or peptide and an agonist, an antibody, or an antagonist. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

Throughout this description, the terms “macromolecule,” “high molecular weight,” “high molecular weight probe,” and similar terminology denote proteins, fusion proteins, peptides, antibodies, bispecific antibodies, diabodies, and the like having a molecular weight greater than about 50,000, preferably greater than about 60,000, and most preferably greater than about 70,000.

Throughout this description, the expression “low molecular weight labeled probe,” and similar terminology, denote chelates, fragments, proteins, peptides, effectors and the like having a molecular weight less than about 10,000, preferably, less than about 8,000 and most preferably less than about 7,500.

Throughout this description, the expression “short lived radionuclide” denotes a radionuclide that loses its efficacy in a relatively short period of time. In a preferred embodiment of the invention, the short lived radionuclide is selected from F-18, Cu-64, and mixtures thereof.

The multispecific macromolecular constructs used with the compositions and methods of embodiments of the present invention can be prepared by techniques known in the art, for example, by utilizing either a known or prepared antibody and modifying its binding sites to bind both an antigenic determinant that functions as a disease marker as well as to an effector, either a radiolabeled effector or an unlabeled effector conjugated to a carrier protein. The antibodies useful in the present invention also can be prepared using conventional techniques. For example, antibodies can be prepared by injection of an immunogen, such as (peptide)n-KLH, wherein KLH is keyhole limpet hemocyanin, and n=1-30, in complete Freund's adjuvant, followed by two subsequent injections of the same immunogen suspended in incomplete Freund's adjuvant into immunocompetent animals, followed three days after an intravenous boost of antigen, by spleen cell harvesting.

Harvested spleen cells then can be fused with Sp2/0-Ag14 myeloma cells and supernatants of the resulting clones cultured and analyzed for anti-peptide reactivity using a direct-binding ELISA. Fine specificity of generated antibodies can be analyzed by using peptide fragments of the original immunogen. These fragments can be readily prepared using an automated peptide synthesizer. For antibody production, enzyme-deficient hybridomas may be isolated to enable selection of fused cell lines. This technique also can be used to raise antibodies to one or more of the R groups used in the low molecular weight probes of the invention. For example, monoclonal mouse antibodies to an In(III)-di-DTPA are known and described in, for example, U.S. Pat. No. 5,256,395.

The multispecific macromolecular constructs used in the present invention preferably are specific to a variety of cell surface or intracellular tumor-associated antigens as marker substances, or to markers of atherosclerosis such as LOX-1, and the like. These markers may be substances produced by a tumor or may be substances that accumulate at a tumor site, on tumor cell surfaces or within tumor cells, whether in the cytoplasm, the nucleus or in various organelles or sub-cellular structures. Among such tumor-associated markers are those disclosed by Herberman, “Immunodiagnosis of Cancer,” in Fleisher ed., The Clinical Biochemistry of Cancer, page 347 (American Association of Clinical Chemists, 1979) and in U.S. Pat. Nos. 4,150,149; 4,361,544; and 4,444,744, the disclosures of which are incorporated by reference herein in their entirety.

Tumor-associated markers have been categorized in a number of categories including oncofetal antigens, placental antigens, oncogenic or tumor virus associated antigens, tissue associated antigens, organ associated antigens, ectopic hormones and normal antigens or variants thereof. Occasionally, a sub-unit of a tumor-associated marker is advantageously used to raise antibodies having higher tumor-specificity, e.g., the beta-subunit of human chorionic gonadotropin (HCG) or the gamma region of carcino embryonic antigen (CEA), which stimulate the production of antibodies having a greatly reduced cross-reactivity to non-tumor substances as disclosed in U.S. Pat. Nos. 4,361,544 and 4,444,744.

Examples of tumor-associated antigens include useful as targets for the compositions of the invention include, but are not limited to, members of the CT antigens (including CT9, CT10, LAGE, MAGE-B5, -B6, -C2, -C3 and -D, HAGE, and SAGE); MAGE, BAGE, and GAGE antigens; melanosome protein antigens; CEA; RU2; Class I HLA-restricted Tumor-specific antigens; bcr-abl; CAP-1; DAM; MART-1/Melan-A; PRAME; PSA; PSM; SART-1; SART-3; and pm1-RARα.

Further tumor-associated antigens which may be targeted using the compositions and methods of the instant invention include, but are not limited to, the anti-carcinoembryonic antigen (“CEA”), the anti-colon-specific antigen-p (“CSAp”), as well as other non-limiting tumor associated antigens including CD19, CD20, CD21, CD22, CD23, CD30, CD74, CD80, HLA-DR, MUC-1, MUC-2, MUC-3, MUC-4, EGF-R, HER2/neu, PAM-4, Bre3, TAG-72 (C72.3, CC49), EGP-1 (e.g., RS7), EGP-2 (e.g., 17-1A and other Ep-CAM targets), LeY (e.g., B3), A3, KS-1, S100, IL-2, T101, necrosis antigens, folate receptors, angiogenesis markers (e.g., VEGF-R, flt-1), tenascin, PSMA, PSA, tumor-associated cytokines, MAGE and/or fragments thereof.

Another marker of interest is transmembrane activator and CAML-interactor (TACI). See Yu et al. Nat. Immunol. 1:252-256 (2000). Briefly, TACI is a marker for B-cell malignancies (e.g., lymphoma). Further it is known that TACI and B cell maturation antigen (BCMA) are bound by the tumor necrosis factor homolog a proliferation-inducing ligand (APRIL). APRIL stimulates in vitro proliferation of primary B and T cells and increases spleen weight due to accumulation of B cells in vivo. APRIL also competes with TALL-I (also called BLyS or BAFF) for receptor binding. Soluble BCMA and TACI specifically prevent binding of APRIL and block APRIL-stimulated proliferation of primary B cells. BCMA-Fc also inhibits production of antibodies against keyhole limpet hemocyanin and Pneumovax in mice, indicating that APRIL and/or TALL-I signaling via BCMA and/or TACI are required for generation of humoral immunity. Thus, APRIL-TALL-I and BCMA-TACI form a two ligand-two receptor pathway involved in stimulation of B and T cell function.

After initially raising antibodies to the targeted tissue, the antibodies can be sequenced and subsequently prepared by recombinant techniques. Humanization and chimerization of murine antibodies and antibody fragments are well known to those skilled in the art. For example, humanized monoclonal antibodies can be produced by transferring mouse complementary determining regions from heavy and light variable chains of the mouse immunoglobulin into a human variable domain, and then, substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, in Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun., 150: 2844 (1993).

Alternatively, fully human antibodies can be obtained from transgenic non-human animals, as described in, for example, Mendez et al., Nature Genetics, 15: 146-156 (1997); U.S. Pat. No. 5,633,425. For example, human antibodies can be recovered from transgenic mice possessing human immunoglobulin loci. The mouse humoral immune system may be humanized by inactivating the endogenous immunoglobulin genes and introducing human immunoglobulin loci. The human immunoglobulin loci are exceedingly complex and comprise a large number of discrete segments which together occupy almost 0.2% of the human genome. To ensure that transgenic mice are capable of producing adequate repertoires of antibodies, large portions of human heavy- and light-chain loci typically are introduced into the mouse genome. This is accomplished in a stepwise process beginning with the formation of yeast artificial chromosomes (YACs) containing either human heavy- or light-chain immunoglobulin loci in germline configuration. Since each insert is approximately 1 Mb in size, YAC construction requires homologous recombination of overlapping fragments of the immunoglobulin loci. The two YACs, one containing the heavy-chain loci and one containing the light-chain loci, preferably are introduced separately into mice via fusion of YAC-containing yeast spheroblasts with mouse embryonic stem cells. Embryonic stem cell clones then can be microinjected into mouse blastocysts. Resulting chimeric males preferably are screened for their ability to transmit the YAC through their germline and then bred with mice deficient in murine antibody production. Breeding the two transgenic strains, one containing the human heavy-chain loci and the other containing the human light-chain loci, creates progeny that produce human antibodies in response to immunization.

Unrearranged human immunoglobulin genes also can be introduced into mouse embryonic stem cells via microcell-mediated chromosome transfer (MMCT); Tomizuka et al., Nature Genetics, 16: 133 (1997). In this methodology, microcells containing human chromosomes are fused with mouse embryonic stem cells. Transferred chromosomes are stably retained, and adult chimeras exhibit proper tissue-specific expression.

As an alternative, an antibody or antibody fragment useful in the present invention may be derived from human antibody fragments isolated from a combinatorial immunoglobulin library; Barbas et al., METHODS: A Companion to Methods in Enzymology 2: 119 (1991), and Winter et al., Ann. Rev. Immunol. 12: 433 (1994). Many of the difficulties associated with generating monoclonal antibodies by B-cell immortalization can be overcome by engineering and expressing antibody fragments in E. coli, using phage display. To ensure the recovery of high affinity, monoclonal antibodies a combinatorial immunoglobulin library usually contains a large repertoire size.

A typical strategy utilizes mRNA obtained from lymphocytes or spleen cells of immunized mice to synthesize cDNA using reverse transcriptase. The heavy- and light-chain genes are amplified separately by PCR and ligated into phage cloning vectors. Two different libraries are produced, one containing the heavy-chain genes and one containing the light-chain genes. Phage DNA is isolated from each library, and the heavy- and light-chain sequences are ligated together and packaged to form a combinatorial library. Each phage contains a random pair of heavy- and light-chain cDNAs and upon infection of E. coli directs the expression of the antibody chains in infected cells. To identify an antibody that recognizes the antigenic tissue of interest, the phage library is plated, and the antibody molecules present in the plaques are transferred to filters. The filters are incubated with radioactively labeled antigen and then washed to remove excess unbound ligand. A radioactive spot on the autoradiogram identifies a plaque that contains an antibody that binds the antigen. Cloning and expression vectors that are useful for producing a human immunoglobulin phage library can be obtained, for example, from STRATAGENE Cloning Systems (La Jolla, Calif.).

A similar strategy can be employed to obtain high-affinity single chain Fv antibody fragment (scFv); Vaughn et al., Nat. Biotechnol., 14: 309-314 (1996). An scFv library with a large repertoire can be constructed by isolating V-genes from non-immunized human donors using PCR primers corresponding to all known VH, Vκ and V80 gene families. Following amplification, the Vκ and Vλ pools are combined to form one pool. These fragments can be ligated into a phagemid vector. The scFv linker, (Gly4, Ser)3, then may be ligated into the phagemid upstream of the VL fragment. The VH and linker-VL fragments can be amplified and assembled on the JH region. The resulting VH-linker-VL fragments then can be ligated into a phagemid vector. The phagemid library can be panned using filters, as described above, or using immunotubes (Nunc; Maxisorp).

Similar results can be achieved by constructing a combinatorial immunoglobulin library from lymphocytes or spleen cells of immunized rabbits and by expressing the scFv constructs in P. pastoris; Ridder et al., Biotechnology, 13: 255-260 (1995). In addition, following isolation of an appropriate scFv, antibody fragments with higher binding affinities and slower dissociation rates can be obtained through affinity maturation processes such as CDR3 mutagenesis and chain shuffling; Jackson et al., Br. J. Cancer, 78: 181-188 (1998); Osbourn et al., Immunotechnology, 2: 181-196 (1996).

Another form of an antibody fragment is a peptide coding for a single CDR. CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells; Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

In one embodiment of the invention, after preparing the antibody that binds or recognizes the tissue of interest, a bispecific antibody can be prepared using techniques known in the art. For example, an anti-CEA tumor antibody and an anti-peptide antibody both can be separately digested with pepsin to their respective F(ab′)2s. The anti-CEA-antibody-F(ab′)2 then can be reduced with cysteine to generate Fab′ monomeric units that can further be reacted with the cross-linker bis(maleimido)hexane to produce Fab′-maleimide moieties. The anti-peptide antibody-F(ab′)2 may be reduced with cysteine and the purified, recovered anti-peptide Fab′-SH reacted with the anti-CEA-Fab′-maleimide to generate the Fab′×Fab′ bi-specific Ab. Alternatively, the anti-peptide Fab′-SH fragment may be coupled with the anti-CEA F(ab′)2 to generate a F(ab′)2×Fab′ construct, or with anti-CEA IgG to generate an IgG×Fab′ bi-specific construct. In one embodiment of the invention, the IgG×Fab′ construct can be prepared in a site-specific manner by attaching the antipeptide Fab′ thiol group to anti-CEA IgG heavy-chain carbohydrate which has been periodate-oxidized, and subsequently activated by reaction with a commercially available hydrazide-maleimide cross-linker. The component antibodies used can be chimerized or humanized by known techniques.

Single chain antibody fragments of an antibody raised against a tissue of interest can be genetically engineered as fusion protein with, e.g., streptavidin, using the techniques disclosed in, for example, Goshorn, S, et al., Cancer Biother. Radiopharm, 16(2):109-123, (2001); Shultz, et al., Cancer Res., 60(23): 6663-69, (2000). The R group for the effector used in the present invention therefore can be any R group capable of binding streptavidin, such as biotin. Biotin-streptavidin pretargeting strategies are well known and described in numerous publications. In the known systems, fusion proteins typically are prepared by genetically engineering an antibody fragment that recognizes a tissue of interest (i.e., one arm of an antibody) and streptavidin (the other arm of the bispecific antibody). Biotin then is conjugated to a chelate using conventional chelators like DOTA, TETA, and DTPA, which in turn is conjugated to a labeling compound. The present invention uses an effector that is distinct from the conventional chelators, and that is capable of binding a short lived radionuclide and an effector capable of binding the multispecific macromolecular construct.

A variety of recombinant methods can be used to produce multispecific macromolecular construct fragments. For example, bi-specific antibodies and antibody fragments can be produced in the milk of transgenic livestock; Colman, A., Biochem. Soc. Symp., 63: 141-147, 1998; and U.S. Pat. No. 5,827,690. Two DNA constructs can be prepared that contain, respectively, DNA segments encoding paired immunoglobulin heavy and light chains. The fragments may be cloned into expression vectors that contain a promoter sequence that is preferentially expressed in mammary epithelial cells. Examples include, but are not limited to, promoters from rabbit, cow and sheep casein genes, the cow α-lactoglobulin gene, the sheep β-lactoglobulin gene and the mouse whey acid protein gene. Preferably, the inserted fragment is flanked on its 3′ side by cognate genomic sequences from a mammary-specific gene. This provides a polyadenylation site and transcript-stabilizing sequences. The expression cassettes may be coinjected into the pronuclei of fertilized, mammalian eggs, that then are implanted into the uterus of a recipient female and allowed to gestate. After birth, the progeny are screened for the presence of both transgenes by Southern analysis. In order for the antibody to be present, both heavy and light chain genes typically must be expressed concurrently in the same cell. Milk from transgenic females can be analyzed for the presence and functionality of the antibody or antibody fragment using standard immunological methods known in the art. The antibody can be purified from the milk using standard methods known in the art.

A chimeric antibody preferably is prepared by ligating the cDNA fragment encoding the mouse light variable and heavy variable domains to fragment encoding the C domains from a human antibody. Because the C domains typically do not contribute to antigen binding, the chimeric antibody will retain the same antigen specificity as the original mouse antibody but will be closer to human antibodies in sequence. Chimeric antibodies still contain some murine sequences, however, and may still be immunogenic. A humanized antibody contains only those mouse amino acids necessary to recognize the antigen. This product is constructed by building into a human antibody framework the amino acids from mouse complementarity determining regions.

Other recent methods for producing bispecific antibodies include engineered recombinant antibodies that have additional cysteine residues so that they crosslink more strongly than the more common immunoglobulin isotypes; FitzGerald et al., Protein Eng. 10(10): 1221-1225, 1997. Another approach is to engineer recombinant fusion proteins linking two or more different single-chain antibody or antibody fragment segments with the needed dual specificities; Coloma et al., Nature Biotech. 15:159-163, 1997. A variety of bi-specific fusion proteins can be produced using molecular engineering. In one form, the bi-specific fusion protein is monovalent, consisting of, for example, a scFv with a single binding site for one antigen and a Fab fragment with a single binding site for a second antigen. In another form, the bi-specific fusion protein is divalent, consisting of, for example, an IgG with two binding sites for one antigen and two scFv with two binding sites for a second antigen. In either case, one of the binding sites is for the tissue of interest and the other is for the effector molecule (radiolabeled and non-radiolabeled) of the present invention.

Functional bi-specific single-chain antibodies, also called diabodies, can be produced in mammalian cells using recombinant methods; Mack et al., Proc. Natl. Acad. Sci., 92: 7021-7025, 1995. For example, diabodies can be produced by joining two single-chain Fv fragments via a glycine-serine linker using recombinant methods. The V light-chain (VL) and V heavy-chain (VH) domains of two antibodies of interest may be isolated using standard PCR methods. The VL and VH cDNAs obtained from each hybridoma are then joined to form a single-chain fragment in a two-step fusion PCR. The first PCR step introduces the (Gly4-Ser)3 linker, and the second step joins the VL and VH amplicons. Each single chain molecule then may be cloned into a bacterial expression vector. Following amplification, one of the single-chain molecules can be excised and sub-cloned into the other vector, containing the second single-chain molecule of interest. The resulting diabody fragment then can be subcloned into a eukaryotic expression vector. Functional protein expression can be obtained by transfecting the vector into chinese hamster ovary (CHO) cells. Bi-specific fusion proteins also can be prepared in a similar manner. Bi-specific single-chain antibodies and bi-specific fusion proteins both can be used in the present invention. Bi-specific fusion proteins linking two or more different single-chain antibodies or antibody fragments also can be produced in a similar manner.

Large quantities of bispecific antibodies and fusion proteins can be produced using E. coli expression systems; Zhenping et al., Biotechnology, 14: 192-196, 1996. A functional bispecific antibody can be produced by the coexpression in E. coli of two “cross-over” scFv fragments in which the VL and VH domains for the two fragments are present on different polypeptide chains. The V light-chain (VL) and V heavy-chain (VH) domains of two antibodies of interest may be isolated using standard PCR methods. The cDNA's then can be ligated into a bacterial expression vector such that the C-terminus of the VL domain of the first antibody of interest is ligated via a linker to the N-terminus of the VH domain of the second antibody. Similarly, the C-terminus of the VL domain of the second antibody of interest may be ligated via a linker to the N-terminus of the VH domain of the first antibody.

The resulting dicistronic operon can then be placed under transcriptional control of a strong promoter, e.g., the E. coli alkaline phosphatase promoter that is inducible by phosphate starvation. Alternatively, single-chain fusion constructs have been successfully expressed in E. coli using the lac promoter and a medium consisting of 2% glycine and 1% Triton X-100; Yang et al., Appl. Environ. Microbiol., 64: 2869-2874, 1998. An E. coli, heat-stable, enterotoxin II signal sequence can be used to direct the peptides to the periplasmic space. After secretion, the two peptide chains associate to form a non-covalent heterodimer that possesses both antigen binding specificities. The bispecific antibody then can be purified using standard procedures known in the art, e.g., Staphylococcal protein A chromatography.

Functional bispecific antibodies and fusion proteins also can be produced in the milk of transgenic livestock, as described above with respect to the methods of making recombinant bispecific antibodies. Functional bispecific antibodies and fusion proteins can also be produced in transgenic plants; Fiedler et al., Biotech., 13: 1090-1093, 1995; Fiedler et al., Immunotechnology, 3: 205-216, 1997. Such production offers several advantages including low cost, large scale output and stable, long term storage. The bispecific antibody fragment, obtained as described above, then can be cloned into an expression vector containing a promoter sequence and encoding a signal peptide sequence, to direct the protein to the endoplasmic recticulum. A variety of promoters can be utilized, allowing the practitioner to direct the expression product to particular locations within the plant. For example, ubiquitous expression in tobacco plants can be achieved by using the strong cauliflower mosaic virus 35S promoter, while organ specific expression can be achieved via the seed specific legumin B4 promoter. The expression cassette may be transformed according to standard methods known in the art, and transformation typically is verified by Southern analysis. Transgenic plants then can be analyzed for the presence and functionality of the bispecific antibody using standard immunological methods known in the art. The bispecific antibody then can be purified from the plant tissues using standard methods known in the art.

Transgenic plants may also facilitate long term storage of bispecific antibodies and fusion proteins. Functionally active scFv proteins have been extracted from tobacco leaves after a week of storage at room temperature. Similarly, transgenic tobacco seeds stored for 1 year at room temperature show no loss of scFv protein or its antigen binding activity.

Functional bispecific antibodies and fusion proteins also may be produced in insect cells; Mahiouz et al., J. Immunol. Methods, 212: 149-160 (1998). Insect-based expression systems provide a means of producing large quantities of homogenous and properly folded bscAb. The baculovirus is a widely used expression vector for insect cells and has been successfully applied to recombinant antibody molecules; Miller, L. K., Ann. Rev. Microbiol., 42: 177 (1988); Bei et al., J. Immunol. Methods, 186: 245 (1995). Alternatively, an inducible expression system can be utilized by generating a stable insect cell line containing the bispecific antibody construct under the transcriptional control of an inducible promoter; Mahiouz et al., J. Immunol. Methods, 212: 149-160 (1998). The bispecific antibody fragment, obtained as described above, then can be cloned into an expression vector containing the Drosphila metallothionein promoter and the human HLA-A2 leader sequence. The construct then can be transfected into D. melanogaster SC-2 cells. Expression can be induced by exposing the cells to elevated amounts of copper, zinc or cadmium. The presence and functionality of the bispecific antibody can be determined using standard immunological methods known in the art, and purified bispecific antibodies can be obtained using standard methods known in the art.

Multivalent target binding proteins also can be used in the invention. Multivalent target binding proteins have been made by cross-linking several Fab-like fragments via chemical linkers (U.S. Pat. Nos. 5,262,524; 5,091,542 and Landsdorp et al. Euro. J. Immunol. 16: 679-83 (1986)). Multivalent target binding proteins also have been made by covalently linking several single chain Fv molecules (scFv) to form a single polypeptide (U.S. Pat. No. 5,892,020). A multivalent target binding protein that is basically an aggregate of scFv molecules has been disclosed in U.S. Pat. Nos. 6,025,165 and 5,837,242, and a trivalent target binding protein comprising three scFv molecules has been described in Krott et al. Protein Engineering 10(4): 423-433 (1997). These binding proteins can be used to target a particular tissue with one of its binding sites, and to target a portion of the low molecular weight labeled molecule of the present invention.

Another composition of the invention that is particularly useful for increasing the success and accuracy of imaging techniques is a blocking agent that comprises, or alternatively consists of, an unlabeled effector that is the same or a similar effector to the radiolabeled effector, conjugated by a linker moiety to a carrier protein or polypeptide. In one embodiment of the invention, the carrier is a macromolecule. In another embodiment of the invention, the carrier is a protein or polypeptide, modified or unmodified DNA/RNA strands, or single strands thereof. In a preferred embodiment of the invention, the carrier is a low immunogenicity carrier. In a particularly preferred embodiment of the invention, the carrier is a non-immunogenic carrier.

After administration of the multispecific macromolecular construct, such as a diabody or bispecific antibody, to a subject, a blocking agent can be used to help clear any residual non-bound entities from circulation. Any blocking agent consistent with this disclosure can be used, including a glycosylated anti-idiotypic Fab′ fragment targeted against the disease targeting arm(s) of the multispecific macromolecular construct. Anti-CEA (MN 14 Ab) × anti-peptide bispecific antibody may be administered and allowed to accrete in disease targets to its maximum extent. To clear residual bispecific antibody, an anti-idiotypic Ab to MN-14, termed WI2, can be administered, preferably as a glycosylated Fab′ fragment. In a non-limiting embodiment of the invention, the blocking agent is bound by the multispecific macromolecular construct in a monovalent manner, while its appended glycosyl residues direct the entire complex to the liver, where rapid metabolism takes place. In a preferred embodiment of the invention, the blocking agent comprises, or alternatively consists of, an unlabeled effector molecule conjugated by a linker moiety to a carrier protein. Suitable linker molecules are set forth infra in the disclosure.

In a non-limiting hypothesis of the invention, the blocking agent is believed to improve the overall characteristics and accuracy of the imaging procedure by allowing unbound multispecific macromolecular construct, such as bispecific antibodies or diabodies, to bind to the blocking agent via the unlabeled effector conjugated to the carrier, thereby helping to prevent non-specific binding of the multispecific macromolecular construct in the subject.

In one embodiment of the invention, the carrier protein to which the unlabeled effector molecule is conjugated is a large protein or polypeptide, and the unlabeled effector is conjugated to the carrier protein so that the effector is exposed on the surface of the carrier protein, thereby allowing for the binding of the multispecific macromolecular construct, such as a bispecific antibody or diabody. It is particularly preferred that the carrier protein be non-immunogenic in the subject. Accordingly, preferred carrier proteins comprise, or alternatively consist of, natural human serum proteins of substantial size. Preferred carrier proteins include, but are not limited to, large, non-immunogenic single chain antibody fragments; human serum albumin or fragments thereof; human transferrin or fragments thereof; DNA/RNA strands; DNA/RNA analog strands; and human and humanized antibodies or immunoglobulins or fragments thereof. Carrier proteins may be derived from natural sources or recombinantly produced.

The blocking agent of the invention has the following general formula:
C-l-R
where C is a carrier such as a protein, polypeptide, macromolecule, DNA/RNA or DNA/RNA analog or chimeric construct, l is a linking ligand capable of linking C to R, and R is an unlabeled effector molecule that is recognized or capable of being bound by a multispecific macromolecular construct. Preferably the carrier C is a non-immunogenic or low immunogenicity carrier, and does not have clinically-relevant binding affinity for molecules located in the human body.

Any effector may function in the invention, provided that R is capable of binding the multispecific macromolecular construct, including those discussed above. For example, R can include but is not limited to, cyclohexyl alanine, DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), and combinations and metal complexes thereof. Preferred R groups are chelates, although any R group having the properties described herein can be used in the invention. In addition, the R groups can be any compound or chelate whereby an antibody was raised against the chelate complexed to yttrium. A particularly preferred R group is represented by the following formula:

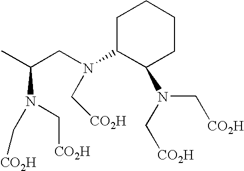

The effector (e.g., chelate or other R group) of the invention also preferably includes a linking moiety to more readily enable conjugation of the carrier protein to the effector. The use of linking moieties is particularly useful in the compositions of the invention, as the linking moiety presents the R group effector molecule in a more accessible fashion for binding and reduces steric hindrances that may influence binding to the R group.

Suitable linking moieties for use in the invention include, but are not limited to, any bi-valent linking moieties. Preferably, the linking moieties include but are not limited to isothiocyanate entities, cyanates, cyanilide, sulfur, oxygen, peptides, thiols, sulfonamides, carboxamides, hydrazinocarbonyl moieties, and combinations thereof. Preferred linking moieties comprise, or alternatively consist of, peptides that may be produced using any number of techniques. In one embodiment of the invention, the linking moiety is a peptide and is produced using recombinant protein production methods as a fusion protein between the linking moiety and the carrier protein. The linking moiety also may be a single covalent bond between a carbon on the R group and the carrier protein or polypeptide.

The radiolabeled effector molecule of the invention has the following formula:
La-l-R
where La is a short-lived labeling agent, l is a linking ligand capable of linking La to R, and R is the effector molecule capable of being bound by the multispecific macromolecular construct.

Radiolabeled effectors are useful compositions in the methods of the invention for enabling the imaging, detection and diagnosis of disease states in a subject. Numerous radiolabels may be used to generate radiolabeled effectors that are useful in imaging and detection. For example, a non-limiting list of radiolabels that may be used to generate radiolabeled effectors include 11C, 13N, 15O, 18F, 52Fe , 62Cu, 64Cu, 67Cu, 67Ga, 68Ga, 86Y, 89Zr, 94mTc, 94Tc, 99mTc, 111In, 123I, 124I, 125I, 131I, 154-158Gd and 175Lu. Particularly preferred radiolabels comprise, or alternatively consist of, F-18, Cu-64 and mixtures thereof.

18F can be obtained from cyclotrons after bombardment of O-18-enriched water with protons. The enriched water containing H-18F can be neutralized with a base having a counter-cation that is any alkylammonium, tetraalkylammonium, alkylphophosphonium, alkylquanidium, alkylamidinium or alkali metal (M), such as potassium, cesium, or other monovalent ions that are strongly chelated to a ligand such as Kryptofix 222 (4,7,13,16,21,24-hexaoxa-1,10-diazabycyclo[8.8.8]hexacosane), such that the resulting alkali metal-ligand complex is freely soluble in organic solvents such as acetonitrile, dimethylsulfoxide, or dimethylformamide. The water can be evaporated off to produce a residue of countercation-18F, which can be taken up in an organic solvent for further use. In general, the counter-cation is selected to enable the fluoride ion to react rapidly in an organic phase with a halogen.

Because fluoride is the most electronegative element, it has a tendency to become hydrated and lose its nucleophilic character. To minimize this, the labeling reaction preferably is performed under anhydrous conditions. For example, fluoride (as potassium fluoride or as a complex with any of the other counter-ions discussed above) can be placed in organic solvents, such as acetonitrile or THF. With the assistance of agents that bind to the counter-cation, such as Kryptofix 2.2.2 (4,7,13,16,21,24-hexaoxa-1,10-diazabicyclo[8.8.8]-hexacosane), the fluoride ion is very nucleophilic in these solvents. The remaining portion of the chelate molecule of the invention then can be added to the solvent and the chelate thereby labeled with the 18F. Using the guidelines provided herein, those skilled in the art are capable of labeling the chelate molecules of the present invention with 18F.

Although potassium is useful as the metal in the counter-cations in accordance with the present invention, cesium is preferred to potassium because cesium is a larger ion with a more diffuse charge. Accordingly, cesium has looser ionic interactions with the small fluoride atom, and therefore does not interfere with the nucleophilic properties of the fluoride ion. For similar reasons, potassium is preferred to sodium, and, in general, the suitability of a lanthanide metal as the metal in the counter-cation in accordance with the present invention increases as you go down the periodic table. Group Ib reagents, such as silver, also are useful as counter-ions in accordance with the present invention. Further, organic phase transfer-type ions, such as tetraalkylammonium salts, also can be used as counter-cations.

Other suitable labeling agents include those that can be bound to the linking ligand (l) tightly enough not to be cleared from circulation under normal circumstances. Thus, the labeling agent will remain attached to the low molecular weight labeled molecule of the present invention.

Any molecule R that is capable of being bound by the multispecific macromolecular construct can be used as the R group. For example, R can include but is not limited to, cyclohexyl alanine, DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), and combinations and metal complexes thereof. Preferred R groups are chelates, although any R group having the properties described herein can be used in the invention. However, for purposes of the invention the R group is preferably the same R group that is used in either the blocking agent or the radiolabeled hapten, or at a minimum at least possesses the same antigenic determinants which enable binding by the multispecific macromolecular construct, such as a bispecific antibody or diabody. In addition, the R groups can be any compound or chelate whereby an antibody was raised against the chelate complexed to yttrium. A particularly preferred R group is represented by the following formula:

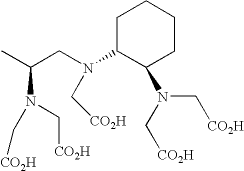

The effector used with the invention also preferably includes a linking moiety to more readily enable conjugation of the label La to the R group. Suitable linking moieties for use in the invention include any bi-valent linking moieties. Preferably, the linking moieties include isothiocyanate entities, cyanates, cyanilide, sulfur, oxygen, peptides, thiols, sulfonamides, carboxamides, hydrazinocarbonyl moieties, and combinations thereof. The linking moiety also may be a single covalent bond between a carbon on the R group and 18F. It is most preferred that the linking group of the present invention, when coupled to fluorine as the La group, is represented by the following formulae: 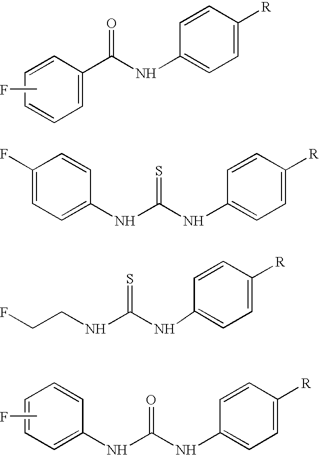
R in the above formulae is a molecule capable of being bound by the multispecific macromolecular construct, such as a bispecific antibody or diabody.

The radiolabeled effector molecules of the present invention can be prepared using conventional synthesis techniques. Those skilled in the art are capable of synthesizing the radiolabeled effector molecules of the invention, using the guidelines provided herein. Certain techniques of radiolabeling some compounds are disclosed, for example, in U.S. Pat. Nos. 5,569,446; 5,308,603; 6,080,384; 5,514,363; and 4,636,380, which are herein incorporated by reference.

The invention is also directed to methods of diagnosing, detecting or imaging a disease or disorder in a subject comprising administering to said subject a multispecific macromolecular construct, such as a bispecific antibody or diabody, having specificity for a tissue specific marker such as a tumor specific antigen, as well as having specificity for an effector (unlabeled or radiolabeled). Following administration of the multispecific macromolecular construct, a period of time is allowed to elapse thereby enabling the multispecific macromolecular construct to bind with specificity to target tissues or sites throughout the subject's body.

Following administration of the multispecific macromolecular construct and after allowing a period of time to elapse for the multispecific macromolecular construct to bind to the target tissue, a blocking agent is administered to the subject wherein said blocking agent comprises, or alternatively consists of, a blocking agent as provided herein. The blocking agent comprises, or alternatively consists of, an unlabeled effector conjugated by a linking moiety to a carrier, such as a protein or polypeptide. In a preferred embodiment of the invention, the carrier protein or polypeptide is a non-immunogenic or low immunogenicity protein or peptide such as an endogenous human serum protein, such as for example, human serum albumin, human transferrin or human immunoglobulin, or fragments thereof.

Following administration of the blocking agent, a period of time is allowed to elapse wherein the blocking agent is bound by circulating or unbound multispecific macromolecular construct through the unlabeled effector conjugated to the carrier. In a nonlimiting hypothesis of the invention, it is believed that the overall success of an imaging procedure is enhanced through the addition of the blocking agent to the method of imaging, by helping to prevent non-specific binding of the multispecific macromolecular construct or to reduce the background signal from circulating or unbound multispecific macromolecular construct that may otherwise yield inconsistent or errant results.

After said period of time allowing binding of the blocking agent by circulating or unbound multispecific macromolecular construct has elapsed, a radiolabeled effector is administered to the subject. Preferably, the radiolabeled effector comprises, or alternatively consists of, the same effector molecule that is conjugated to the carrier (such as a protein or polypeptide), conjugated to a linker molecule and a radiolabel agent. At a minimum, the labeled and unlabeled effector molecules share at least one cross-reactive antigenic determinant, thereby allowing for binding to both by the multispecific macromolecular construct. In one embodiment of the invention, the radiolabeled effector binds to the multispecific macromolecular construct bound to the target tissue of interest, and is imaged using techniques discussed herein and known in the art. Preferably, the radiolabeled effector molecule is labeled with 18F.

Depending on the particular label that has been attached to the radiolabeled effector molecules, the appropriate imaging technique is employed to image the targeted tissue. For example, when 18F is used as the labeling agent PET imaging is conducted and the targeted tissue is labeled.

The imaging method can be used as a diagnostic to detect the presence of a diseased or unwanted tissue; can be used to detect the extent of growth of a diseased or unwanted tissue; and can be used to image throughout the body. In addition, the imaging method can be repeated over a number of days to provide a quantitative assessment of the degree of growth or spreading of the targeted tissue if applicable, such as for example a malignant tissue.

In one embodiment of the invention, the multispecific macromolecular construct is an antibody or a fragment thereof, preferably a humanized antibody or fragment thereof, raised against a tumor-associated antigen.

Tissue-specific antibodies against cells, for example bone marrow cells, expressing CD34 or CD74, as well as antibodies against non-malignant diseased biomarkers, such as macrophage antigens of atherosclerotic plaques (e.g., anti-CD74 antibodies), are well known in the art, as are antibodies against bacteria, viruses and parasites. It is noted again that the foregoing disclosure of various antigens or biomarkers described previously herein as useful for raising antibodies having specificity against them is merely exemplary, and is in no way intended to limit the present invention.

The invention also encompasses compositions comprising, or alternatively consisting of, the multispecific macromolecular construct such as a bispecific antibody or diabody, the blocking agent and the radiolabeled effector of the invention, as well as a kit for imaging a targeted tissue. The kit preferably comprises three separate compositions; one including the radiolabeled effector molecule of the invention, another including the blocking agent, and the last containing a multispecific macromolecular construct that is capable of binding to both the radiolabeled effector and the unlabeled effector conjugated to the carrier (such as a protein or polypeptide) as well as to the targeted tissue. The radiolabeled and unlabeled effector molecules useful in the compositions may be those used for imaging or those used for therapy. Additionally, the kit may comprise instructions for the administration of the individual compositions of the kit to a subject.

The order of addition of the multispecific macromolecular construct and the blocking agent to the targeted tissue is not critical, and may even be administered in a relatively close proximity of time, provided that both are administered prior to the administration of the radiolabeled effector molecule which functions as the imaging agent.

Embodiments of the invention may be practiced in ways other than those particularly described in the foregoing description and examples. Numerous modifications and variations of the invention are possible in light of the above teachings and, therefore, are within the scope of the appended claims.

The entire disclosure of each document cited (including patents, patent applications, journal articles, abstracts, manuals, books, or other disclosures) in the Background of the Invention, Detailed Description, and Examples is herein incorporated by reference in their entireties.

The invention now will be explained with reference to the non-limiting examples.

### EXAMPLES

### Example 1

### Clinical Imaging of Colon Cancer

A bispecific antibody is used as the multivalent macromolecular construct in the instant example. A bispecific antibody is injected into a colon cancer patient and allowed to accumulate over a period of several hours to a few days at colon cancer lesions expressing CEA (carcinoembryonic antigen).

A blocking agent is injected, which associates itself rapidly to the bispecific antibody in circulation by binding, making the bispecific antibody substantially unavailable for subsequent binding. By virtue of its large size (the carrier molecule is a 100 kDa DNA strand), the blocking agent penetrates lesions slowly enough that it will not block a significant portion of bispecific antibodies at the lesion site.

After some time (minutes to a few hours), 10 mCi of an F-18 labeled peptide-based effector is injected, which quickly penetrates the lesions and binds to the bifunctional antibody, while being prevented from binding to the bifunctional antibody in circulation by the blocking agent.

Standard PET imaging is performed at 1 to 4 hours post-effector injection. A diagnosis is made from the PET image. At least an approximate ten-fold improvement in target-to-blood effector molecule concentration at the target location achieved by using the compositions and methods of the invention, when compared to a substantially similar imaging method lacking administration of a blocking agent, will be indicative of success.

### Example 2

### Preclinical Imaging in Animal Models

A tumor-bearing mouse is injected with a bispecific antibody that accumulates over a period of several hours to a few days at colon cancer xenograft expressing CEA (carcinoembryonic antigen).

A blocking agent is subsequently injected, which associates itself rapidly to the bispecific antibody in circulation, making it unavailable for later binding. By virtue of its large size (the carrier molecule is a 100 kDa DNA strand), the blocking agent penetrates the xenograft slowly enough that it will not block a significant portion of bispecific antibodies bound at the xenograft site.

After some time (minutes to a few hours), 200 μCi of a Cu-64 labeled peptide-based effector is injected, which quickly penetrates the xenograft and binds to the bifunctional antibody, while being prevented from binding to the bifunctional antibody in circulation by the blocking agent.

PET imaging is performed at 1 to 4 hours post-effector injection. Xenograft effector uptake is measured from the PET image. The tumor-bearing mouse is treated with a therapeutic agent that should affect CEA expression.

After a few days, the tumor-bearing mouse is injected with a bispecific antibody that accumulates over a period of several hours to a few days at colon cancer xenograft expressing CEA (carcinoembryonic antigen). A blocking agent is injected, which associates itself rapidly to the bispecific antibody in circulation, making it unavailable for later binding. By virtue of its large size (the carrier molecule is a 100 kDa DNA strand), the blocking agent penetrates the xenograft slowly enough that it will not block a significant portion of bispecific antibodies at the xenograft site.

After some time (minutes to a few hours), 200 μCi of a Cu-64 labeled peptide-based effector is injected, which quickly penetrates the xenograft and binds to the bifunctional antibody, while being prevented from binding to the bifunctional antibody in circulation by the blocking agent. PET imaging is performed a 1 to 4 hours post-effector injection.

The results of the first PET image are compared with the second PET image in order to assess whether CEA expression decreases due to the action of the therapeutic agent.

While the invention has been described with reference to particularly preferred examples and embodiments, those skilled in the art will appreciate that various modifications may be made to the invention without departing from the spirit and scope thereof.

What is claimed is:

1. A blocking agent comprising an effector conjugated to a carrier molecule by a linking moiety.

2. The blocking agent of claim 1, wherein said carrier molecule is a macromolecule.

3. The blocking agent of claim 1, wherein said carrier molecule is selected from human serum albumin or fragments thereof, human transferrin or fragments thereof, DNA/RNA strands, DNA/RNA analog strands, human antibodies, humanized antibodies or fragments thereof, and chimeras of the same.

4. The blocking agent of claim 3, wherein the antibody fragment comprises a single-chain antibody fragment.

5. The blocking agent of claim 3, wherein the antibody fragment does not have clinically-relevant binding affinity for molecules located in the human body.

6. The blocking agent of claim 1, wherein the effector is unlabeled.

7. The blocking agent of claim 1, wherein the effector is selected from cyclohexyl alanine, DTPA, 1,4,7-triaza-cyclononane-N,N′,N″-triacetic acid (NOTA), p-bromoacetamido-benzyl-tetraethylaminetetraacetic acid (TETA), 1,4,7,10-tetraazacyclododecanetetraacetic acid (DOTA), and combinations and metal complexes thereof.

8. The blocking agent of claim 1, wherein the linking moiety is a peptide.

9. The blocking agent of claim 8, wherein the peptide linking moiety is produced recombinantly as a fusion protein with the carrier protein or carrier polypeptide.

10. The blocking agent of claim 1, wherein the linking moiety is selected from isothiocyanate entities, cyanates, cyanilide, sulfur, oxygen, thiols, sulfonamides, carboxamides, hydrazinocarbonyl moieties, and combinations thereof.

11. The blocking agent of claim 1, wherein the effector is a chelate.

12. A method of diagnosing, detecting or imaging a disease or disorder of a mammal, comprising:
administering to said mammal a multispecific macromolecular construct having binding specificity for both a mammalian tissue and an effector;
administering to the mammal a blocking agent comprising an effector to which said multispecific macromolecular construct has specificity linked to a carrier by a linking moiety; administering to the mammal a radiolabeled effector; and imaging the mammal.

13. The method of claim 12, wherein the mammal is a human.

14. The method of claim 12, wherein the mammal is a mouse.

15. The method of claim 12, wherein the mammal is a rat.

16. The method of claim 12, wherein said blocking agent comprises an effector conjugated to a carrier molecule by a linking moiety.

17. The method of claim 16, wherein said carrier molecule is a low immunogenicity macromolecule.

18. The method of claim 17, wherein said carrier molecule is selected from human serum albumin or fragments thereof, human transferrin or fragments thereof, and human antibody or fragments thereof.

19. The method of claim 18, wherein the antibody fragment comprises a single-chain antibody fragment.

20. The method of claim 18, wherein the antibody fragment does not have clinically relevant binding affinity for target molecules located in the human body.

21. The method of claim 12, wherein the effector associated with the blocking agent is unlabeled.

22. The method of claim 12, wherein the effector is selected from cyclohexyl alanine, DTPA, 1,4,7-triaza-cyclononane-N,N′,N″-triacetic acid (NOTA), p-bromoacetamido-benzyl-tetraethylaminetetraacetic acid (TETA), 1,4,7,10-tetraazacyclododecanetetraacetic acid (DOTA), and combinations and metal complexes thereof.

23. The method of claim 16, wherein the linking moiety is a peptide.

24. The method of claim 23, wherein the peptide linking moiety is produced recombinantly as a fusion protein with the carrier protein or carrier polypeptide.

25. The method of claim 16, wherein the linking moiety is selected from isothiocyanate entities, cyanates, cyanilide, sulfur, oxygen, thiols, sulfonamides, carboxamides, hydrazinocarbonyl moieties, and combinations thereof.

26. The blocking agent of claim 12, wherein the effector is a chelate.

27. The method of claim 12, wherein a period of time is provided after administration of the multispecific macromolecular construct to allow the multispecific macromolecular construct to bind to the target molecules of interest.

28. The method of claim 12, wherein a period of time is provided after administration of the blocking agent to allow the blocking agent to bind to the multispecific macromolecular construct.

29. The method of claim 12, wherein the multispecific macromolecular construct is a bispecific antibody.
